(19) United States
(12) Patent Application Publication
(10) Pub. No.: US 2025/0218545 A1
(43) Pub. Date: Jul. 3, 2025

(54) LARGE LANGUAGE MODEL DRIVEN DATA AUGMENTATION FOR PROTEIN MACHINE LEARNING

(71) Applicant: X Development LLC, Mountain View, CA (US)

(72) Inventor: Federico Vaggi, Seattle, WA (US)

(73) Assignee: X Development LLC, Mountain View, CA (US)

(21) Appl. No.: 18/397,412

(22) Filed: Dec. 27, 2023

(51) Int. Cl. G16B 40/00; G16B 30/00

(52) U.S. Cl. CPC G16B 40/00; G16B 30/00

(57) ABSTRACT

A method for training a machine learning model (MLM) to predict the activity of a protein is described herein. In an example, a method involves accessing a set of training data comprising labeled examples with known activity levels. A large language model is used to generate synthetic examples of each labeled example by incorporating each possible amino acid (AA) mutation at each AA position in the labeled example and predicting the probability each AA mutation has of replacing the original AA. Based on a predetermined cutoff, a subset of negative synthetic examples that comprises at least one AA mutation with the lowest probability of being incorporated are selected. An augmented training dataset is generated and a MLM is trained, using the training data and the augmented training data set, by performing iterative operations to find a set of parameters that jointly minimize the sum of at least two loss functions.

### FIELD

The present disclosure relates in general to machine learning techniques in the field of protein engineering, and more particularly, to leveraging large language model driven negative data augmentation of functional protein sequences to efficiently create negative training data with high contrast for training a machine learning model.

### BACKGROUND

Proteins are macromolecules built from one or more long chains of amino acids (polypeptide chains) that fold into 3-dimensional structures that determine the function or activity of the protein. The amino acid sequence of a protein is determined by the “genetic code” stored in the DNA sequence of the protein-encoding gene. The genetic code of a gene serves as an instruction manual that informs a cell how to synthesize a specific protein. A two-step process is used to convert DNA into a protein. First DNA is transcribed into RNA and second, RNA (more specifically mRNA) is translated into a protein. During this process, the genetic code is preserved from DNA to RNA and is interpreted in a series of triplets or three nucleotide units known as codons. Given that there are four nucleotides found in RNA (A, U, G, and C), there are 64 possible triplet combinations, thus 64 possible codons. Each codon specifies a particular amino acid; however, because there are only 20 naturally occurring amino acids, almost every amino acid can be signaled by multiple codons.

To form the 3-dimensional structure of their protein, the chain of amino acids interact with each other to first fold into secondary structures (i.e., alpha helices, beta sheets, beta turns, and random coils). After, the secondary structures will arrange and interact with each other to fold into what is known as a tertiary structure, or a single protein subunit. For many proteins, their function or activity is dependent on the assembly of multiple subunits (either the same or different) coming together to form a quaternary structure. At this point, most proteins are considered to be in their native structure and depending on their function may be active or inactive.

In a cell, proteins participate in nearly every biological process (e.g., enzymes, scaffolds, signaling molecules, etc.), thus folding into their correct 3-dimensional structure is essential to proper function and activity. Failure to fold into its native structure generally results in an inactive protein that is degraded by the cell. In some instances, failure to fold is caused by alterations (i.e., mutations) in the amino acid sequence that cannot be tolerated because they are in essential sites for protein self-assembly. In other instances, the amino acid mutation may result in a misfolded protein that is not degraded and instead has toxic effects on the cell as seen in several neurodegenerative disorders. Further, in other examples, amino acid mutations can have no effect on protein function, or they can enhance the activity of a protein. This later example of enhancing protein function is of particular interest to researchers and clinicians for the potential therapeutic opportunities enhancing protein function presents. However, identifying beneficial mutations is experimentally slow and expensive given that for every protein sequence, the search space comprises 20 possible points of mutations, where N equals the number of amino acids in the protein sequence.

### SUMMARY

Techniques (e.g., computer implemented methods, systems, and computer-program products) are described herein for exponentially increasing the amount of functional training data available (at no additional experimentation cost) for training a machine learning model to predict protein activity. By leveraging the negative protein activity data generated from large language models (LLMs), negative data augmentation is used to generate a negative training dataset with high contrast between the functional protein sequences and the LLM generated negative protein sequences. Accordingly, a machine learning model is trained to identify mutations to the functional protein sequences that are highly likely to have significantly less activity. In so doing, the amount of time, resources, and expenses are considerably reduced as researchers can avoid generating, validating, and characterizing protein sequences predicted to significantly reduce protein function.

In various embodiments, a computer-implemented method is provided comprising: accessing a set of training data comprising labeled examples that are protein sequences with known activity levels; generating, using a large language model, synthetic examples by incorporating each possible amino acid mutation into the labeled examples; selecting, based on a predetermined cutoff, a negative subset of the synthetic examples that comprise at least one amino acid mutation with a lowest probability of being incorporated into a labeled example; pairing the negative subset of the synthetic examples with respective labeled examples to generate an augmented training dataset comprising pairs of contrastive examples; training, using the set of training data and the augmented training dataset, a machine learning model to predict activity of a protein sequence, where the training is an iterative process that comprises: (a) feeding a portion or an entirety of the set of training data into the machine learning model, performing computations to generate predictions, comparing the predictions to the known activity levels using a loss function in order to quantify error or loss of the machine learning model, (b) feeding a portion or an entirety of the augmented training dataset into the machine learning model, performing computations to generate predictions, comparing the predictions for contrast using a contrastive function in order to quantify error or loss of the machine learning model, (c) adjusting parameters of the machine learning model that jointly minimizes a sum of the loss function and the contrastive function, and (d) repeating (a), (b), and (c) for a number of iterations or epochs; and outputting the trained machine learning model.

In some embodiments, the large language model is pretrained using a masked marginal approach training scheme.

In some embodiments, generating the synthetic examples further comprises: in an iterative process starting at the first amino acid position of the labeled example, introducing a mask token, incorporating each possible amino acid mutation into the masked position, predicting the probabilities for each possible amino acid mutation being incorporated based on the surrounding amino acids of the labeled example, repeating the iterative process at the second and subsequent amino acid positions of the labeled example, outputting the synthetic examples, where each of the synthetic examples comprises at least one possible amino acid mutation; sorting, based on the probabilities for each possible amino acid mutation, the synthetic examples; and pairing the negative subset of synthetic examples with the labeled example; and labeling the labeled example and the negative subset of the synthetic examples with labels indicating the labeled example has higher activity compared to the negative subset of synthetic examples.

In some embodiments, each possible amino acid mutation comprises substitutions, deletions, insertions, or any combination thereof that involves at least one original amino acid in the labeled examples.

In some embodiments, each possible amino acid mutation comprises performing a substitution.

In some embodiments, each possible amino acid mutation comprises substituting the at least one original amino acid, inserting one or more amino acids, or any combination thereof with one or more amino acids selected from a list including alanine, cysteine, aspartic acid, glutamic acid, phenylalanine, glycine, histidine, isoleucine, arginine, lysine, leucine, methionine, asparagine, proline, glutamine, serine, threonine, valine, tryptophan, and tyrosine.

In some embodiments, the negative subset of the synthetic examples are highly likely to have reduced activity compared to the labeled examples.

In some embodiments, a system is provided that includes one or more data processors and a non-transitory computer readable storage medium containing instructions which, when executed on the one or more data processors, cause the one or more data processors to perform part or all of one or more methods or processes disclosed herein.

In some embodiments, a computer-program product is provided that is tangibly embodied in a non-transitory machine-readable storage medium and that includes instructions configured to cause one or more data processors to perform part or all of one or more methods disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by embodiments and optional features, modification and variation of the concepts herein disclosed may be resorted to by those skilled in the art, and that such modifications and variations are considered to be within the scope of this invention as defined by the appended claims.

### BRIEF DESCRIPTION OF THE DRAWINGS

The present invention will be better understood in view of the following non-limiting figures, in which:

FIG. 1 shows a block diagram illustrating a system for implementing techniques for performing negative data augmentation to train a machine learning model to predict protein sequences with low activity in accordance with various embodiments.

FIG. 2 illustrates negative data augmentation by the large language model to generate training data for a machine learning in accordance with various embodiments.

FIG. 3 is a flowchart illustrating a process for training a machine learning model in accordance with various embodiments.

FIG. 4 is a flowchart illustrating how a trained machine learning model is implemented in accordance with various embodiments.

### DETAILED DESCRIPTION

The ensuing description provides preferred exemplary embodiments only, and is not intended to limit the scope, applicability or configuration of the disclosure. Rather, the ensuing description of the preferred exemplary embodiments will provide those skilled in the art with an enabling description for implementing various embodiments. It is understood that various changes may be made in the function and arrangement of elements without departing from the spirit and scope as set forth in the appended claims.

Specific details are given in the following description to provide a thorough understanding of the embodiments. However, it will be understood that the embodiments may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

Also, it is noted that individual embodiments may be described as a process which is depicted as a flowchart, a flow diagram, a data flow diagram, a structure diagram, or a block diagram. Although a flowchart or diagram may describe the operations as a sequential process, many of the operations may be performed in parallel or concurrently. In addition, the order of the operations may be re-arranged. A process is terminated when its operations are completed but could have additional steps not included in a figure. A process may correspond to a method, a function, a procedure, a subroutine, a subprogram, etc. When a process corresponds to a function, its termination may correspond to a return of the function to the calling function or the main function.

### I. Introduction

When seeking to design or discover novel proteins, protein engineering relies on one of the most fundamental concepts in evolutionary biology: the process of natural selection where favorable mutations are selected and propagated through the population while deleterious mutations are removed. In nature, mutations arise randomly and their effect on protein structure, and ultimately function, depends on where in the protein the mutations occur. Estimates suggest that nearly 50-80% of all amino acid residues in a given protein sequence can be changed without significantly altering the structure. On the other hand, mutations that arise in evolutionary conserved regions, in connection to binding sites, or that do impact protein structure (e.g., introduction of charged amino acids into buried sites or mutations that disrupt beta-sheets) more often have a severe impact on phenotype, leading to increased susceptibility for disease. Therefore, the challenge in protein engineering lies in being able to efficiently filter through and remove the 50-80% of silent mutations and the deleterious mutations to focus efforts on creating therapeutically beneficial proteins.

Traditionally, protein engineering techniques rely on labor intensive and costly experimental design and characterization methods to identify potentially useful proteins. For example, if all 20 naturally occurring amino acids are tested at N positions in a protein sequence, the total number of possible mutations is 20^{N}. Thus, to experimentally design, characterize, and validate a short protein sequence comprising only 100 amino acids, 20¹⁰⁰ individual experiments would need to be conducted, amounting to an obtusely infeasible task. In an effort to circumnavigate this obstacle, machine learning approaches and deep-learning networks have emerged as useful protein engineering tools.

Most commonly, LLMs are trained using unsupervised techniques, such as masked training, where protein sequence data without labels are used. However, LLMs trained this way perform poorly with regards to predicting mutations that increase the protein activity. In order to train a LLM to predict functional proteins or even proteins with increased activity, a supervised training approach is needed. Supervised LLM training involves adding “tags” or “labels” to the protein sequence data that incorporate information on taxonomy, function, and location to make inferences on protein structure and activity. Unfortunately, these types of labels are not included for the majority of the available protein sequence due to the cumbersome nature of generating such data. Protein sequence datasets that do include experimentally validated functional data are small in size and thus insufficient to train a machine learning model to accurately make predictions on protein function. Further, as described above, the experimental capacity to generate a sufficient training data set is much smaller than the total number of possible mutations that require testing. Due to these limitations, training a supervised machine learning model on functional data to predict amino acid mutations that would improve protein activity is not possible.

Contrastive learning is an alternative to typical cardinal supervised machine learning, which uses a cost function (e.g., cardinal loss) to map an event or values of one or more variables onto a real number intuitively representing some cost associated with the event. Contrastive learning can be applied in unsupervised, semi-supervised, and supervised machine learning to learn the general features of a dataset either without or with labels by teaching the model which data points are similar or different. The objective is to minimize the distance between positive pairs (e.g., instances from the same sample) and maximize the distance between negative pairs (e.g., instances from different samples). For example, contrast loss can be used for optimizing protein/enzyme activity without requiring the exact activity readouts. Instead, contrastive losses would only require knowing that a particular sequence A is more active than another sequence B. However, contrast learning still requires an abundance of training examples having similar and different activity levels in order to learn the high-level features for distinguishing between similar and different data points.

To address these challenges and others, techniques are described herein that leverage large language model driven data augmentation of functional protein sequences to efficiently create the negative data points (training examples) for training a supervised machine learning model with a supervised loss function and a contrastive objective function. Generally, LLMs are not as good at predicting high activity mutations: most of the correlation in zero shot experiment scenarios is in identifying mutations that decrease activity, relative to a baseline. As discussed above, experimental data is expensive and slow to collect. When scientists do collect experimental data, there is a desire to focus on collecting positive examples with high activity not negative examples with low or reduced activity. Consequently, the techniques described herein focus on the use of LLMs to efficiently create the negative examples. The techniques include obtaining a functioning protein with a given sequence and utilizing a protein LLM to identify, by a masked marginal approach, edits (i.e., mutations) to the protein sequence that significantly decrease the likelihood of the protein remaining functional or having a similar protein activity. Advantageously, these techniques can be implemented as a form of data augmentation, making it fully compatible with most deep machine learning training pipelines. For example, from a single protein that has 50 amino acids, 950 synthetic contrasts (50*19 possible substitutions from remaining amino acids) can be generated by the LLM. Applying this technique to an entire database of protein sequences of various lengths will significantly increase the number of contrasts that can be used to train a deep learning model to make inferences on protein activity. Moreover, these techniques significantly increase the dataset size at zero to little additional experimentation cost because they are being synthetically generated using the protein LLM.

In one particular aspect, a computer-implemented method is provided for that comprises accessing a set of training data comprising labeled examples that are protein sequences with known activity levels; generating, using a large language model, synthetic examples by incorporating each possible amino acid mutation into the labeled examples; selecting, based on a predetermined cutoff, a negative subset of the synthetic examples that comprise at least one amino acid mutation with a lowest probability of being incorporated into a labeled example; pairing the negative subset of the synthetic examples with respective labeled examples to generate an augmented training dataset comprising pairs of contrastive examples; training, using the set of training data and the augmented training dataset, a machine learning model to predict activity of a protein sequence, where the training is an iterative process that comprises: (a) learning model, performing computations to generate predictions, comparing the predictions to the known activity levels using a loss function in order to quantify error or loss of the machine learning model, (b) feeding a portion or an entirety of the augmented training dataset into the machine learning model, performing computations to generate predictions, comparing the predictions for contrast using a contrastive function in order to quantify error or loss of the machine learning model, (c) adjusting parameters of the machine learning model that jointly minimizes a sum of the loss function and the contrastive function, and (d) repeating (a), (b), and (c) for a number of iterations or epochs; and outputting the trained machine learning model.

As used herein, when an action is “based on” something, this means the action is based at least in part on at least a part of the something. As used herein, the terms “similarly”, “substantially,” “approximately” and “about” are defined as being largely but not necessarily wholly what is specified (and include wholly what is specified) as understood by one of ordinary skill in the art. Further, the terms “similarly”, “substantially,” “approximately,” or “about” may be substituted with “within [a percentage] of” what is specified, where the percentage includes 0.1, 1, 5, and 10 percent.

### II. Computing Environment

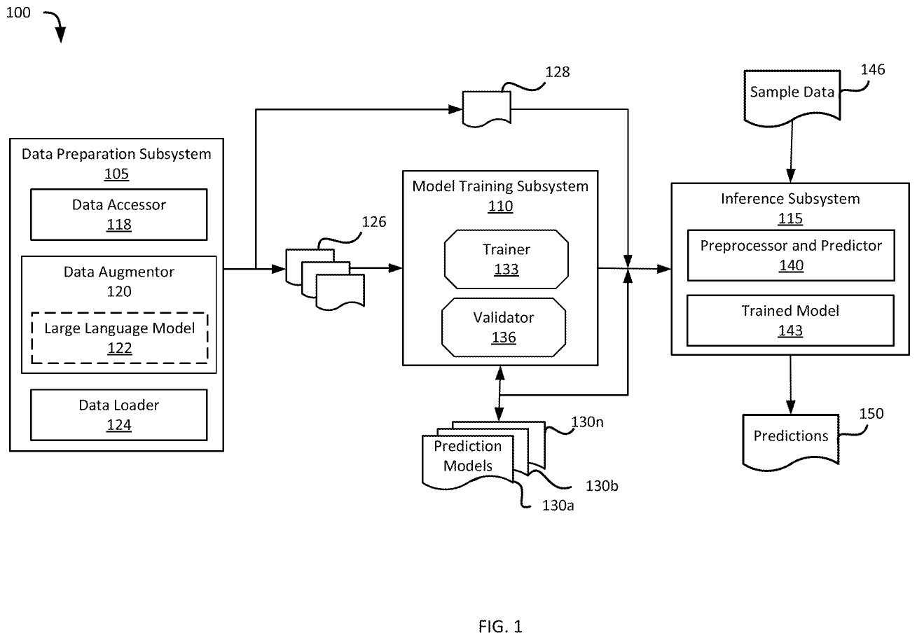

FIG. 1 shows a system 100 for training a machine learning model to predict the activity of a protein sequence in accordance with various embodiments. As shown in FIG. 1, the system 100 comprises several subsystems: a data preparation subsystem 105 to obtain, generate, and store datasets, a model training subsystem 110 to build and train models, and an inference subsystem 115 for implementing trained machine learning models.

The data preparation subsystem 105 is configured to access data (e.g., protein sequences), use the data to perform large language model driven negative data augmentation to create training/validation and testing datasets, and prepare the data to be used by the other subsystems. Data accessor 118 facilitates the process of accessing data. The data may be accessed from a data storage structure such as a database, a research and development laboratory, a publicly available database, or any other method in which protein sequences may be acquired. The data may include sequences of proteins, peptides, polypeptides, or peptide portions whose activity level has been predetermined through experimental validation. As used herein, the terms proteins, peptides, polypeptides, or peptide portions are used broadly and interchangeably herein to mean two or more amino acids linked by a peptide bond. Further, all four terms apply to naturally occurring amino acid polymers. It should be recognized that the term peptide is not used herein to suggest a particular size or number of amino acids comprising the molecule.

The term “amino acid” refers to any monomeric unit that can be incorporated into a peptide, polypeptide, or protein. Amino acids include naturally occurring α-amino acids and their stereoisomers. “Stereoisomers” of a given amino acid refer to isomers having the same molecular formula and intramolecular bonds but different three-dimensional arrangements of bonds and atoms (e.g., an L-amino acid and the corresponding D-amino acid).

Naturally occurring amino acids are those encoded by the genetic code. Naturally-occurring α-amino acids include, without limitation, alanine (Ala), cysteine (Cys), aspartic acid (Asp), glutamic acid (Glu), phenylalanine (Phe), glycine (Gly), histidine (His), isoleucine (Ile), arginine (Arg), lysine (Lys), leucine (Leu), methionine (Met), asparagine (Asn), proline (Pro), glutamine (Gln), serine (Ser), threonine (Thr), valine (Val), tryptophan (Trp), tyrosine (Tyr), and combinations thereof. Stereoisomers of a naturally-occurring α-amino acids include, without limitation, D-alanine (D-Ala), D-cysteine (D-Cys), D-aspartic acid (D-Asp), D-glutamic acid (D-Glu), D-phenylalanine (D-Phe), D-histidine (D-His), D-isoleucine (D-Ile), D-arginine (D-Arg), D-lysine (D-Lys), D-leucine (D-Leu), D-methionine (D-Met), D-asparagine (D-Asn), D-proline (D-Pro), D-glutamine (D-Gln), D-serine (D-Ser), D-threonine (D-Thr), D-valine (D-Val), D-tryptophan (D-Trp), D-tyrosine (D-Tyr), and combinations thereof.

Training a machine learning model, in particular a protein LLM, requires a corpus of diverse data to produce accurate and high-quality results that capture the nuances of the protein language Although there is a plethora of protein sequencing data available, there are very few and small datasets available where the activity of a protein is directly measured. As a result, the number of labeled examples (e.g., protein sequences with known activity levels) for training data obtained by data accessor 118, are insufficient to train a supervised machine learning model to predict protein sequences with improved activity levels. To overcome this challenge, the labeled examples of protein sequences with experimentally determined activity levels are input into data augmenter 120. Data augmenter 120 comprises protein large language model (LLM) 122, which is pretrained to generate edited protein sequences (synthetic examples), based on the input (e.g., labeled examples) protein sequences, by incorporating amino acid mutations. In so doing, protein LLM 122 drives a process referred to as data augmentation, which is a process of artificially expanding the sample size of a dataset (e.g., labeled examples of protein sequences with known activity levels), based on prior knowledge about consistent/unchanging properties of a sample (e.g., a protein sequence) to produce additional examples. This technique is typically used in instances where there is an insufficient amount of data to complete a task, such as training a machine learning model. Further, data augmentation can generate “positive” examples of how a task should be solved or “negative” examples of how a task should not be solved. For example, in the instance where positive examples of protein sequences with experimentally determined activity levels are limited, negative synthetic examples of protein sequences with a high likelihood of having low activity may be generated.

In general, protein LLMs 122 are much better at predicting amino acid mutations that render the protein nonfunctional compared to amino acid mutations that improve protein activity. Therefore, data augmenter 120 is configured to use protein LLM 122 driven negative data augmentation to generate a dataset of synthetic examples for each of the input labeled examples. Accordingly, the synthetic examples can have an activity level greater than, equal to, or lower than their corresponding labeled example. In some embodiments, the synthetic examples are negative synthetic examples that have an activity level lower than their corresponding labeled example. To increase the likelihood that the negative synthetic examples are highly likely to have lower activity compared to their corresponding labeled example, a predetermined threshold is used to select the bottom percentage of the synthetic examples. The predetermined threshold is based on the relationship between the protein sequence and the activity level of each labeled example. Furthermore, the more stringent the predetermined threshold is (e.g., bottom 5% vs. bottom 10%) the greater the likelihood that the synthetic examples are true negative examples (e.g., synthetic example has lower activity level compared to corresponding labeled example) compared to false negative examples (e.g., synthetic example has greater than or equal activity level compared to corresponding labeled example). In other words, the negative synthetic examples may additionally comprise examples that have greater than or equal activity levels compared to their corresponding labeled example. In some instances, the negative synthetic examples may not comprise examples that have greater than or equal activity levels compared to their corresponding labeled example.

As described herein, “likely”, “more likely”, or “highly likely” refers to a greater than 50% chance (e.g., 51%, 55%, 60%, 70%, 80%, 90%, and 100%). Furthermore, the term “significant” or “significantly” refers to a statistically significant result that has been predicted as unlikely to have occurred by chance alone according to a predetermined threshold probability referred to as a significance level (e.g., p-values, false discovery rate (FDR), q-values, etc. as values less than 0.05, 0.01, 0.001, 0.0001)

As described herein, the term “activity” refers to the biological function of a protein. In some instances, the activity of a protein may be enhanced or reduced due to one or more amino acid mutations. The terms “enhanced or “reduced” refers to either an increase or decrease (respectively) in the activity of the labeled and/or synthetic examples (e.g., about 30%, 40%, 50%, 60%, 70%, 80%, and 100% increase or decrease in the activity). As way of example, and not limitation, protein activity can describe the enzymatic activity or binding affinity of a protein on its target, or the expression or stability of the protein.

The protein LLM 122 may be pretrained using various training schemes known in the art that result in a LLM with sufficient zero shot performance to generate synthetic examples. Examples, and without limitation, may include masked pretraining, tokenization, variational autoencoder pretraining, autoregressive, etc. In some embodiments, protein LLM 112 is pretrained based on a masked marginal objective so that it will (i) generate synthetic examples that comprise at least one amino acid mutation (e.g., substitutions, insertions, deletions of amino acids) and (ii) predict the probability of each possible amino acid mutation that may be incorporated into at least one position of the labeled example, based on the context of the surrounding amino acids. In other words, the protein LLM 122 will introduce a mask token at each amino acid position in the labeled example and record the predicted probabilities for all 20 amino acids at that position. The predicted probability for each possible amino acid mutation represents how likely or unlikely each amino acid mutation is to be incorporated into the labeled example. In some instances, the amino acid mutations are substitutions where the original amino acid in the labeled example is replaced with one of the remaining 19 amino acids. For example, if a labeled example comprises 200 amino acids, starting at the first amino acid position, the protein LLM 122 generates 19 new synthetic examples by substituting the original amino acid for each of the 19 remaining amino acids (e.g., amino acid mutations). For each synthetic example, the protein LLM 122 will predict the probability each amino acid mutation is incorporated into the labeled example. The protein LLM 122 repeats this process for the second amino acid position through the 200^{th} amino acid position in the labeled example, generating 19 new synthetic examples at each position. This iterative process results in the generation of 3,800 (19×200) synthetic examples from one 200 amino acid long protein sequence. Moreover, the synthetic examples generated by LLM 122 comprise protein sequences that have greater than, equal to, and lower activity levels compared to their corresponding labeled example protein sequence. In some cases, the new synthetic examples may comprise more than one amino acid mutation, exponentially increasing the number of synthetic examples. As described herein, the synthetic examples can comprise an amino acid sequence having at least 90%, 91%, 92%, 93%, 94%, 95%, 96%, 97%, 98%, 99%, 99.5%, and 99.9% sequence identity with the labeled example.

Once data augmenter 120 and protein LLM 122 finish generating synthetic examples for each labeled example input into protein LLM 122, all the labeled examples and synthetic examples are processed by data loader 124 to generate an augmented training dataset. Data loader 124 is configured to (i) sort the synthetic examples (e.g., ascending or descending order) based on the predicted probabilities of each amino acid mutation, (ii) select a subset of the synthetic examples that comprise at least one amino acid mutation with the lowest probability of being incorporated into the labeled example (i.e., negative synthetic examples), (iii) pair the selected negative subset of synthetic examples with their corresponding labeled example, and (iv) label the labeled example as having higher activity compared to its corresponding negative synthetic examples. Data loader 124, selects the synthetic examples with at least one amino acid mutation that is the least likely to be incorporated into the labeled example, as those mutations are more likely to be significantly deleterious to protein function. Selection is based on a predetermined cutoff (e.g., bottom 5% or 10%) to ensure that the augmented training dataset comprises the worst performing negative synthetic examples. Pairing corresponding labeled-negative synthetic examples ensures that during model training, only comparisons in protein activity level between labeled-negative synthetic example pairs are learned. For example, the LLM 122 generates a first set of negative synthetic examples for a first labeled example and a second set of negative synthetic examples for a second labeled example. All that is known is that the first set of negative synthetic examples are likely to be lower in activity compared to their first labeled example. It is unknown if the first set of negative synthetic examples are lower in activity compared to the second labeled example or the second set of negative synthetic examples. Labeling involves adding a “tag” or “label” to the labeled-negative synthetic example pairs, where the labeled example is labeled as having higher activity (compared to its corresponding negative synthetic examples) and the paired negative synthetic examples are labeled as having lower activity (compared to their corresponding labeled example). Finally, data loader 124, will output an augmented training dataset that comprises labeled pairs of comprising pairs of contrastive examples (e.g., labeled-negative synthetic examples) that are accessed by the model training subsystem 110.

Prior to model training and testing, the labeled examples (obtained from data accessor 118) and the augmented training data (prepared by data loader 124) may be split into training and validation datasets 126 so that the system can train and test prediction models 130a-130n (‘n’ represents any natural number). The splitting of the labeled examples and augmented training dataset may be performed randomly (e.g., a 90/10% or 70/30%) or the splitting may be performed in accordance with a more complex validation technique such as K-Fold Cross-Validation, Leave-one-out Cross-Validation, Leave-one-group-out Cross-Validation, Nested Cross-Validation, or the like to minimize sampling bias and overfitting. In addition to training and validation datasets, the data may also be split into a testing data set 128 to be used in the inference subsystem 115 to test the trained model on data it has never seen before.

The model training subsystem 110 comprises two systems: a trainer 133 and a validator 136 for training and validating prediction models 130 to be used by the other subsystems, such as the model inference subsystem 115 for a given task (e.g., predicting the activity of a protein based on its amino acid sequence). The prediction models 130 can be any machine learning model that can be optimized by contrastive loss function, such as any neural network architectures known in the art, for example feed-forward networks, residual networks, convolutional neural networks, recurrent neural networks, etc. In further embodiments, the prediction models 130, are supervised models that can be optimized by contrastive loss functions.

Trainer 133 and validator 136 are part of a machine learning operationalization framework comprising hardware such as one or more processors (e.g., a CPU, GPU, TPU, FPGA, the like, or any combination thereof), memory, and storage that operates software or computer program instructions (e.g., TensorFlow, PyTorch, Keras, and the like) to execute arithmetic, logic, input and output commands for the random forest classifier model. Specifically, trainer 133 performs iterative operations of training that involve inputting portions of training data 126 into prediction models 130 to find a set of model parameters (e.g., weights and/or biases) that minimizes or maximizes an objective function (e.g., a loss function, a cost function, a contrastive loss function, etc.). The portions of training data 126 may be augmented as described with respect to the data augmenter 120. The objective function can be constructed to measure the difference between the outputs inferred using the models and the ground truth annotated to the samples using the labels. For example, for a supervised learning-based model, the goal of the training is to learn a function “h( )” (also sometimes referred to as the hypothesis function) that maps the training input space X to the target value space Y, h:X→Y, such that h(x) is a good predictor for the corresponding value of y. Various different techniques may be used to learn this hypothesis function. In some machine learning algorithms such as a neural network, this is done using back propagation. The current error is typically propagated backwards to a previous layer, where it is used to modify the weights and bias in such a way that the error is minimized or maximized. The weights are modified using the optimization function. Optimization functions usually calculate the error gradient, i.e., the partial derivative of the objective function with respect to weights, and the weights are modified in the opposite direction of the calculated error gradient. For example, techniques such as back propagation, random feedback, Direct Feedback Alignment (DFA), Indirect Feedback Alignment (IFA), Hebbian learning, and the like are used update the model parameters in such a manner as to minimize or maximize this objective function. This cycle is repeated until the minimum or maximum of the objective function is reached.

In some instances, the objective function is a contrastive loss function. A contrastive loss function measures how well the model 130 can contrast between similar and dissimilar data points, where similar points are given positive labels and dissimilar points are given negative labels. Canonical pairwise contrastive loss will minimize the distance between a pair of data points if they are similar or maximize the distance if they are dissimilar. For example, a prediction model 130 trained on negative augmented data will maximize the distance or contrast between the data points being compared (e.g., a functional labeled example and negative synthetic examples with deleterious amino acid mutations). Additionally, or alternatively, the model 130 may be trained with a triplet contrastive loss function where an anchor data point is compared to one similar and one dissimilar data point. In this instance, the contrastive loss function will minimize the distance between the anchor point and the similar data point and maximize the distance between the anchor point and the dissimilar data point.

In some instances, the objective function is a loss function that will measure how well the model 130 performs at predicting the actual activity level of the labeled examples from the set of training data. More specifically, trainer 133 may input portions of training data 126 that were taken from the labeled examples and perform computations to generate a predicted activity level. Then the machine learning model compares the predicted activity level to the ground truth label activity level using the loss function to quantify error or loss.

It should be understood that trainer 133 may perform the contrastive training and the loss function training in any order. As way of example, trainer 133 may first train the machine learning model using the augmented training data and contrastive loss function and then train using labeled examples and the loss function. In another example, trainer 133 may first train the machine learning model using labeled examples and the loss function and then using the augmented training data and contrastive loss function. In further embodiments, the machine learning model may be trained jointly or simultaneously using the augmented training data and contrastive loss function and the labeled examples and the loss function. For example, a portion of the labeled examples are feed into the machine learning model for it to generate predicted activity levels for the labeled examples. Then using the loss function, comparing the predicted activity level to the ground truth label activity level to quantify error or loss. Moreover, positions of the augmented training data are also feed into the machine learning model. Again the machine learning model will make predictions on how similar or different the paired labeled-negative synthetic examples are using the contrastive function to quantify error or loss. Because the machine learning is trained using both datasets simultaneously, its parameters are updated to jointly minimize the sum of the loss function and the contrastive function. This training process may be repeated until for any number of iterations.

Trainer 133 also performs the process of selecting hyperparameters, using an optimization algorithm, to find the model parameters that correspond to the best fit between prediction and actual outputs. Example optimization algorithms include a stochastic gradient descent algorithm or a variant thereof such as batch gradient descent or mini-batch gradient descent. The hyperparameters are settings that can be tuned or optimized to control the behavior of the prediction model 130. Most models explicitly define hyperparameters that control different aspects of the models such as memory or cost of execution. However, additional hyperparameters may be defined to adapt a model to a specific scenario. For example, the hyperparameters may include the number of hidden units of a model, the learning rate of a model, the convolution kernel width, the number of kernels for a model, the number of graph connections to make during a lookback period, the maximum depth of a tree in a random forest, a minimum sample split, a maximum number of leaf nodes, a minimum number of leaf nodes, and the like.

Once a set of model parameters are identified, the model has been trained and is then tested or validated using the validation datasets 126 by validator 136. The validation process includes iterative operations of inputting the validating datasets 126 into the prediction models 130 using a validation technique such as K-Fold Cross-Validation, Leave-one-out Cross-Validation, Leave-one-group-out Cross-Validation, Nested Cross-Validation, or the like to fine tune the hyperparameters and ultimately find the optimal set of hyperparameters. Once the optimal set of hyperparameters are obtained, a reserved set of testing data 128, from the initial splitting of the preprocessed data, are input into trained model 143 to obtain output (in this example, predictions as to whether the input protein sequence comprises amino acid mutations that would reduce protein activity), and the output is evaluated versus ground truth values using correlation techniques such as Bland-Altman method and the Spearman's rank correlation coefficients and calculating performance metrics such as the error, accuracy, precision, recall, receiver operating characteristic curve (ROC), etc.

The model training subsystem 110 outputs trained models 143 with an optimized set of model parameters and hyperparameters for use in the model inference subsystem 115. The model inference subsystem 115 generates an inference phase prediction provided to users using a preprocessor and predictor 140 and the trained model 143. For example, the preprocessor and predictor 140 executes processes for inputting sample data 146 (e.g., a protein sequence of interest with unknown activity levels and synthetic examples with amino acid mutations) into a trained model 143. Then the trained model 143 will output predictions 150 as to whether the synthetic examples have lower activity compared to the labeled example.

The preprocessor and predictor 140 are part of the machine learning operationalization framework comprising hardware such as one or more processors (e.g., a CPU, GPU, TPU, FPGA, the like, or any combination thereof), memory, and storage that operates software or computer program instructions (e.g., Application Programming Interfaces (APIs), Cloud Infrastructure, Kubernetes, Docker, TensorFlow, Kuberflow, Torchserve, and the like) to execute arithmetic, logic, input and output commands for executing a machine learning model in a production environment. In some instances, the preprocessor and predictor 140 implement deployment of the model using a cloud platform such as Amazon Web Services (AWS), Google Cloud Platform (GCP), and Microsoft Azure. A cloud platform makes machine learning more accessible, flexible, and cost-effective while allowing developers to build and deploy the model faster.

### III. Negative Data Augmentation

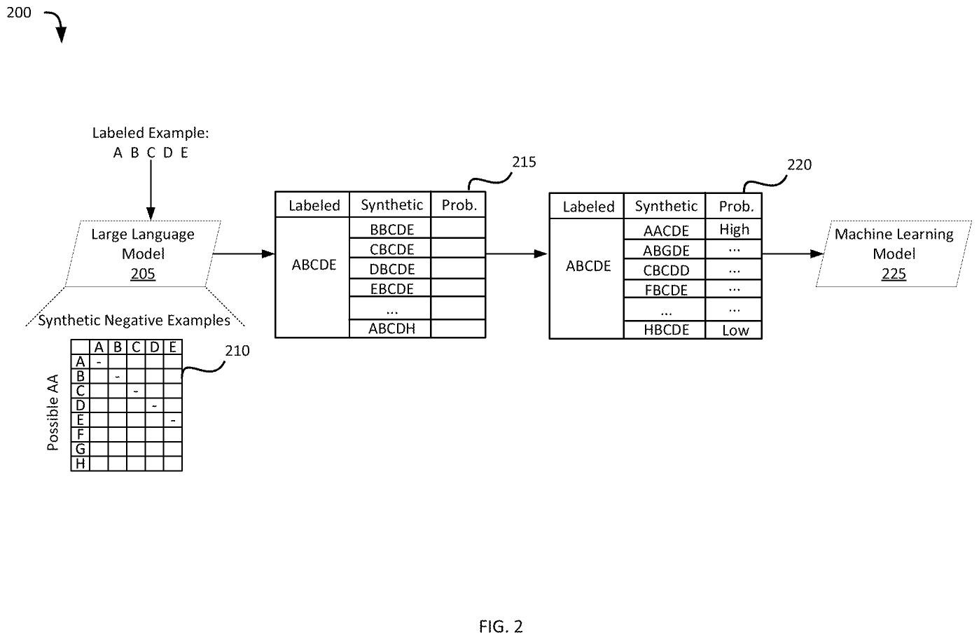

FIG. 2 illustrates process 200 for LLM driven negative data augmentation to generate training data for a machine learning model in accordance with various embodiments. As described above, data augmentation is the process of artificially increasing the size of a dataset with positive, negative, or any combination thereof. For example, negative data augmentation may be used to create in domain negative examples based on a limited database of positive examples (e.g., protein sequences labeled with activity level). Synthetic protein sequences are synthesized in silico using a protein LLM 205 (the same protein LLM described in FIG. 1) that is pre-trained to use a masked marginal scheme to incorporate each possible amino acid mutation at each possible amino acid position of a labeled example. Masked marginal training involves the use of a token to mask each amino acid position in the labeled example. Then, the protein LLM 205 will incorporate each possible amino acid mutation into the masked position and predict the probability each amino acid mutation has of replacing the original amino acid of the labeled example. Possible amino acid mutations can include deletions, insertions, substitutions, or any combination thereof that involves at least one original amino acid in the labeled example. In the event of a deletion, one or more amino acids may be removed from the labeled example, generating synthetic examples that comprise at least 90% sequence identity with the labeled example. When the amino acid mutation is an insertion, one or more amino acids may be added to the labeled example, generating synthetic examples that comprise at least 90% sequence identity with the labeled example. In the event of a substitution, one or more amino acids may be replaced by a different amino acid, generating synthetic examples that comprise at least 90% sequence identity with the labeled example. Moreover, the protein LLM 205 may further incorporate any combination of deletions, insertions, or substitutions to generate synthetic examples comprising at least 90% sequence identity with the labeled example. In some embodiments, each possible amino acid mutation are substitutions, and the synthetic examples may comprise one or more amino acid substitutions. In any of the events described above, deleted, inserted, or substituted amino acids may be selected from a list of amino acids that include alanine, cysteine, aspartic acid, glutamic acid, phenylalanine, glycine, histidine, isoleucine, arginine, lysine, leucine, methionine, asparagine, proline, glutamine, serine, threonine, valine, tryptophan, and tyrosine.

With respect to FIG. 2, it should be understood that the letters ‘A’, ‘B’, ‘C’, ‘D’, and ‘E’, are place holders for any of the 20 naturally occurring amino acids including: alanine, cysteine, aspartic acid, glutamic acid, phenylalanine, glycine, histidine, isoleucine, arginine, lysine, leucine, methionine, asparagine, proline, glutamine, serine, threonine, valine, tryptophan, and tyrosine and are not intended to represent actual amino acid or protein sequences. Moreover, it should also be understood that more than one labeled example may be input into the protein LLM 205.

Process 200 begins with labeled example (e.g., ABCDE) being input into a pretrained protein LLM 205. Starting at the first amino acid position, protein LLM 205 (i) masks the first amino acid and introduces each possible amino acid mutation (e.g., substitutions, insertions, or deletions) that may be incorporated and (ii) records a predicted probability for each possible amino acid mutation representing how likely the mutation is to be incorporated into the original labeled example. In some instances, the amino acid mutation is a substitution where the original amino acid in the labeled example is replaced with each of the remaining possible amino acids. As way of illustration, chart 210 shows this process where columns represent the original amino acids in the labeled example and rows represent each possible amino acid (AA) substitution that may be made. A dash line indicates a substitution of the same amino acid as the labeled example and would thus not be considered as a new synthetic example. For example, the first amino acid ‘A’ in the labeled example can be replaced with amino acids ‘B’, ‘C’, ‘D’, ‘E’, ‘F’, ‘G’, and ‘H’, creating 7 new synthetic examples comprising one amino acid mutation (i.e., a substitution) at the first position. Exemplary synthetic examples are illustrated in chart 215 where the first amino acid ‘A’ is replaced with amino acid ‘B’ (and subsequent amino acids) generating the synthetic example BBCDE along with a predicted probability (Prob.) that amino acid ‘B’ will replace ‘A’. Then, protein LLM 205 masks the second position and will repeat the process of incorporating each possible amino acid mutation at the second position (i.e., ‘A’, ‘C’, ‘D’, ‘E’, ‘F’, ‘G’, and ‘H’), creating 7 new synthetic examples comprising one amino acid mutation at the second position for a total of 14 synthetic examples from the single labeled example. Protein LLM 205 will continue to iteratively mask, substitute, and output synthetic examples for the subsequent amino acid positions in the labeled example.

Although not explicitly illustrated, it should be understood that each synthetic example may comprise one or more amino acid mutations, where the one or more amino acid mutations can comprise substitutions, deletions, insertions, or any combination thereof. In addition, the synthetic examples generated from protein LLM 205 can comprise protein sequences with greater than, equal to, and lower activity levels compared to their corresponding labeled example. The protein LLM 205 will record a predicted probability for each of the one or more amino acid mutations. By way of example, a synthetic example may comprise a first amino acid mutation (e.g., a deletion) with a low/high predicted probability for incorporation and a second amino acid mutation (e.g., an insertion) with a low/high predicted probability for incorporation. In other instances, a synthetic example may comprise a first amino acid mutation (e.g., a substitution) with a high predicted probability for incorporation and a second amino acid mutation (e.g., a substitution) with a low predicted probability for incorporation. A high predicted probability indicates that the new amino acid(s) is very likely to replace the original amino acid(s) (e.g., a silent mutation), whereas a low predicted probability indicates that the new amino acid(s) is very unlikely to replace the original amino(s) (e.g., deleterious mutation).

Once the synthetic examples are generated, they are sorted based on their predicted probability that the new amino acid(s) will replace the original amino acid(s) (see Table 220). A predetermined cutoff (e.g., the bottom 5% or 10% of sequences) is used to select a negative subset of the synthetic examples that have at least one amino acid mutation with the lowest probability of being incorporated into its labeled example. The predetermined cutoff represents a threshold that ensures confidence that the negative subset of synthetic examples are much more likely to comprise mutations that are significantly deleterious to protein activity. In other words, there may be some instances where the negative subset of synthetic examples can include protein sequences with greater than or equal activity levels, in addition to protein sequences with lower activity levels, as their paired labeled example. Finally, the negative subset of synthetic examples are paired with their respective labeled examples to generate an augmented training dataset that comprising pairs of contrastive examples to train machine learning model 225.

### IV. Training a Machine Learning Model

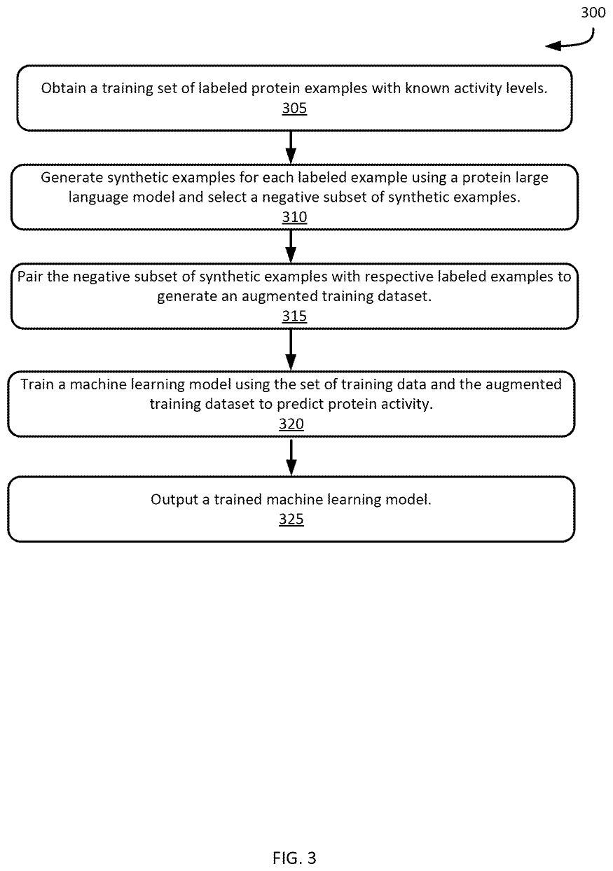

FIG. 3 is a flowchart illustrating a process for training a machine learning model in accordance with various embodiments. The processing depicted in FIG. 3 may be implemented in software (e.g., code, instructions, program) executed by one or more processing units (e.g., processors, cores) of the respective systems, hardware, or combinations thereof (e.g., the intelligent selection machine). The software may be stored on a non-transitory store medium (e.g., on a memory device). The method presented in FIG. 3 and described below is intended to be illustrative and non-limiting. Although FIG. 3 depicts the various processing steps occurring in a particular sequence or order, this is not intended to be limiting. In certain alternative embodiments, the steps may be performed in some different orders, or some steps may also be performed in parallel.

Starting at box 305, a set of training data comprising labeled examples is accessed. The labeled examples comprise protein sequences with activity labels that were previously determined experimentally. In some instances, there may be insufficient quantities of labeled examples with known activity levels to create a labeled set of training data, where the label reflects activity level. To overcome this, synthetic examples for each labeled example are generated that comprise at least one amino acid mutation (e.g., substitutions, insertions, deletions, or any combination thereof).

At box 310, the synthetic examples are generated by inputting the labeled examples into a protein LLM to incorporate each possible amino acid mutation into the labeled examples. More specifically, the synthetic examples can be generated using an iterative process that starts at the first amino acid position of the labeled example. The protein LLM introduces a mask token, incorporates each possible amino acid mutation into the masked position, and predicts the probability for each possible amino acid mutation of being incorporated at a masked position of the labeled example (process described in detail with respect to FIG. 2). This iterative process is repeated at the second and subsequent amino acid positions of the labeled example. In some instances, the synthetic examples will comprise one or more of each possible amino acid mutation. Once all the synthetic examples are generated, they are output and sorted based on their predicted probabilities for each amino acid mutation. When more than one amino acid mutation is incorporated, the synthetic examples may be sorted based on the amino acid mutation with the lowest probability of being incorporated. In general, protein LLMs predict amino acid mutations or edits that are detrimental to protein function rather than beneficial, therefore many of the synthetic examples are likely to have reduced activity compared to their corresponding labeled example. To take advantage of this characteristic, a stringent predetermined cutoff (e.g., bottom 5% or bottom 10%) is used to select a negative subset of synthetic examples that are highly likely to have significantly reduced activity compared to its corresponding labeled example.

At box 315, the negative synthetic examples are paired with their respective labeled examples to generate an augmented training dataset comprising pairs of contrastive examples. Further, the pairs of contrastive examples are labeled with “tags” or “labels” to indicated that the labeled examples have higher activity compared to their respective negative synthetic examples.

At box 320, a machine learning model undergoes training, using the set of training data (from block 305) and the augmented training dataset (from block 315), to predict the activity of a protein sequence. In an iterative process, training comprises feeding a portion or an entirety of the set of training data into the machine learning model. The machine learning model performs computations to generate predictions (e.g., activity level of the labeled examples) and compare those predictions to the ground truth labels using a loss function in order to quantify error or loss of the machine learning model. Alternatively, additionally, or simultaneously, a portion or an entirety of the augmented training dataset is feed into the machine learning model. The machine learning model performs computations to generate predictions (e.g., contrasts or distances) and compare those predictions for contrast using a contrastive function in order to quantify error or loss of the machine learning model. The parameters of the machine learning model are adjusted so that the parameters jointly minimize the sum of the loss function and the contrastive function. This process is repeated for a number of iterations or epochs until a predefined number of iterations or epochs is reached, or when the model's performance stops improving. This prevents the model from overfitting and ensures it generalizes well to unseen data.

More specifically, the contrastive function is configured to maximize the distances between the labeled examples and their corresponding negative synthetic examples. The larger the distance, the more likely the machine learning model is to predict that the negative synthetic example has reduced activity compared to its labeled example. Compared to other loss functions that require labels describing exact readouts (e.g., activity), contrastive loss functions use generalized labels to determine how similar or dissimilar one or more data points may be. For example, that labeled example A has higher activity compared to negative synthetic example B or that one sample is labeled positive while another sample is labeled negative. On the other hand, the loss function is configured to measure a difference between (i) predicted outputs inferred for each labeled example in the training data, and (ii) labels that provide ground truth information for each labeled example in the training data, and where the ground truth information provides experimentally validated activity levels for the protein sequences in the training dataset. The closer the prediction is to the ground truth label, the more likely the machine learning model is to accurately predict the activity level of the labeled examples.

Finally, at box 325, training has concluded, and a trained machine learning model is output that predicts the protein activity of the negative synthetic examples, relative to their corresponding labeled example. In other words, the trained machine learning model can predict, based on mutations/edits made to a labeled example, whether the mutated/edited protein sequence is likely to have reduced activity level compared to the original labeled example. The trained machine learning model may be provided to a machine learning operationalization framework comprising hardware such as one or more processors (e.g., a CPU, GPU, TPU, FPGA, the like, or any combination thereof), memory, and storage that operates deployment tools including software or computer program instructions (e.g., Application Programming Interfaces (APIs), Cloud Infrastructure, Kubernetes, Docker, TensorFlow, Kuberflow, Torchserve, and the like) to execute arithmetic, logic, input and output commands for executing the trained machine learning model in a production environment. In some instances, the deployment tools implement deployment of the fine-tuned decoder model using a cloud platform such as Amazon Web Services (AWS), Google Cloud Platform (GCP), and Microsoft Azure. A cloud platform makes machine learning more accessible, flexible, and cost-effective while allowing developers to build and deploy the fine-tuned decoder model faster.

### V. Implementation of a Trained Machine Learning Model

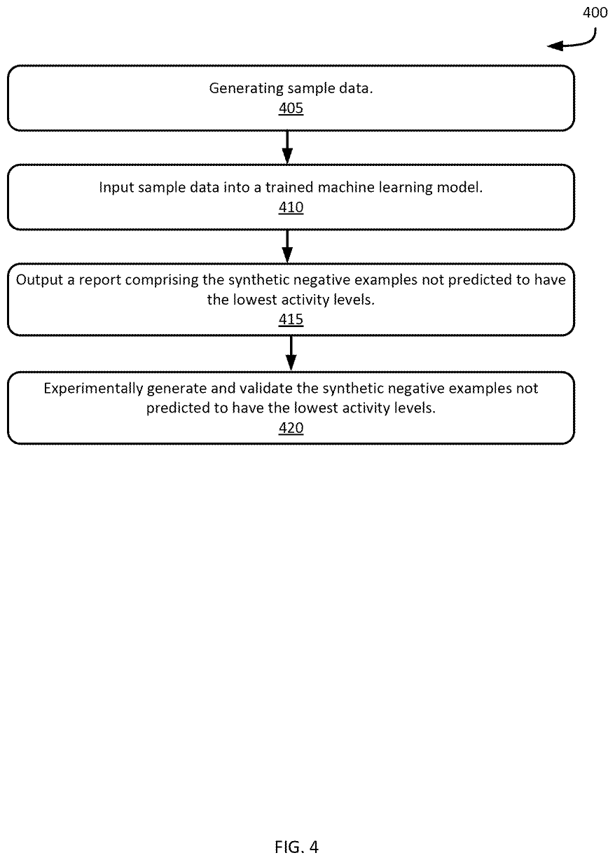

FIG. 4 is a flowchart illustrating process 400 for how a trained machine learning model that predicts protein activity may be used in a research, clinical or therapeutic setting in accordance with various embodiments. The processing depicted in FIG. 4 may be implemented in software (e.g., code, instructions, program) executed by one or more processing units (e.g., processors, cores) of the respective systems, hardware, or combinations thereof (e.g., the intelligent selection machine). The software may be stored on a non-transitory store medium (e.g., on a memory device). The method presented in FIG. 4 and described below is intended to be illustrative and non-limiting. Although FIG. 4 depicts the various processing steps occurring in a particular sequence or order, this is not intended to be limiting. In certain alternative embodiments, the steps may be performed in some different orders, or some steps may also be performed in parallel.

Starting at block 405, sample data is generated. In some instances, the sample data can comprise an amino acid sequence for a protein of interest and synthetic examples comprising amino acid mutations being considered for experimental validation. In other instances, the sample data can comprise an amino acid sequence of a protein of interest and synthetic examples generated by the protein LLM that are based on the protein of interest. In some cases, the activity level of the protein of interest has not been experimentally validated and is unknown. Further, the synthetic examples may comprise at least one amino acid mutation (e.g., substitutions, insertions, deletions, or any combination thereof).

At block 410, the sample data are input into a trained machine learning model to predict whether the synthetic examples have reduced activity compared to the protein sequence of interest. The trained machine learning model uses the predicted probabilities of the one or more amino acid mutations in the synthetic examples to predict if those one or more amino acid mutations are associated with significantly reduced protein activity.

At block 415, the trained machine learning model outputs a report comprising those synthetic examples predicted to have the lowest activity level. The report may be used to filter out synthetic examples that should not be experimentally generated and validated due to their predicted low activity levels. In other words, the report may comprise protein sequences highly likely to encode non-functional proteins. Additionally or alternatively, the trained machine learning model can output a report that comprises those synthetic examples not predicted to have significantly lower activity levels.

At block 420, experimental methods for generating and validating the synthetic examples output from the trained machine learning model are performed. Methods for generating the one or more desired amino acid mutations may include, but are not limited to, site-directed mutagenesis of a plasmid/vector. As described herein, a “plasmid/vector” refers to a circular nucleic acid construct that can be single stranded or double stranded and able to accommodate the necessary experimental elements (e.g., transgene, promoter, selectable marker, etc.). For example, a plasmid/vector may comprise a transgene (e.g., the gene encoding the protein of interest without any mutations), at least one selectable marker (e.g., an antibiotic, reporter, etc.), at least one promoter (e.g., a CMV promoter), and an origin of replication. As described herein, a “plasmid/vector” comprising a transgene is also referred to as a ‘plasmid-gene construct’.

Initially, the plasmid-gene construct undergoes restriction enzyme digest and generates a digested plasmid-gene construct. During this process, a restriction enzyme (e.g., EcoRI, BamHI, Dpn1, etc.) makes a single unique cut at or near its specific recognition site within the transgene. This will generate cut sites with either overhangs (also referred to as sticky ends) or blunt ends, depending on the restriction enzyme used. Preferably, the restriction enzyme generates overhangs.

After restriction enzyme digestion, a small sequence of nucleotides, referred to as an ‘insert’, is mixed with the digested plasmid-gene construct at a ratio sufficient enough that the ‘insert’ is ligated and recombined (e.g., incorporated) into the digested plasmid-gene construct, generating a plasmid-mutated gene construct. The ‘insert’ comprises a nucleotide sequence that encodes the desired amino acid mutation and overhangs that are complementary to the overhangs on the digested plasmid-gene construct that facilitate the incorporation of the ‘insert’ into the plasmid-gene construct. As used herein, the term “nucleic acid or nucleotide” refers to naturally occurring deoxyribonucleic acids (DNA) or ribonucleic acids (RNA) and polymers thereof in either single- or double-stranded form.

Following insertion of the ‘insert’, the plasmid-mutated gene construct is transformed, or introduced, into bacterial cells (e.g., E. coli) via methods such as, without limitation, lipofection, electroporation, microinjection, heat shock, or viral vector. The transformed bacteria are then platted onto an agar dish treated with the same antibiotic as the antibiotic resistant gene (e.g., kanamycin, ampicillin, gentamicin, neomycin, etc.) comprised in the plasmid. Those bacteria cells that successfully up-took the plasmid-mutated gene construct will grow on the antibiotic treated agar medium and are selected for confirmatory sequencing to ensure the desired mutation was incorporated. Once confirmed by sequencing, the selected bacteria cells comprising the plasmid-mutated gene construct with the desired amino acid mutation are cultured in cell media that supports cell growth until the desired concentration of bacterial cells are produced. The bacteria cells are then collected, and protein isolation methods are used to isolate the protein of interest for experimental validation of its activity. Any protein isolation method known in the art may be used to isolate the protein of interest.

The experimental methods used to validate the activity of the protein are dependent on the function/activity of the protein. For example, if the activity of the protein is enzymatic (e.g., inhibitory, activating, modifying, cleaving, etc.) methods may include analyzing the rate, kinetics, product production, substrate consumption, etc. Enzyme assays can include, without limitation, spectrophotometric, fluorometric, calorimetric, chemiluminescent, etc. In another example, the activity of the protein is related to binding a target, in which case enzyme assays can include, without limitation, yeast-two hybrids, co-immunoprecipitation, enzyme-linked immunosorbent assay, etc.

### VI. Additional Considerations

Specific details are given in the above description to provide a thorough understanding of the embodiments. However, it is understood that the embodiments can be practiced without these specific details. For example, circuits can be shown in block diagrams in order not to obscure the embodiments in unnecessary detail. In other instances, well-known circuits, processes, algorithms, structures, and techniques can be shown without unnecessary detail in order to avoid obscuring the embodiments.

Implementation of the techniques, blocks, steps and means described above can be done in various ways. For example, these techniques, blocks, steps and means can be implemented in hardware, software, or a combination thereof. For a hardware implementation, the processing units can be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, other electronic units designed to perform the functions described above, and/or a combination thereof.

Also, it is noted that the embodiments can be described as a process which is depicted as a flowchart, a flow diagram, a data flow diagram, a structure diagram, or a block diagram. Although a flowchart can describe the operations as a sequential process, many of the operations can be performed in parallel or concurrently. In addition, the order of the operations can be re-arranged. A process is terminated when its operations are completed but could have additional steps not included in the figure. A process can correspond to a method, a function, a procedure, a subroutine, a subprogram, etc. When a process corresponds to a function, its termination corresponds to a return of the function to the calling function or the main function.

Furthermore, embodiments can be implemented by hardware, software, scripting languages, firmware, middleware, microcode, hardware description languages, and/or any combination thereof. When implemented in software, firmware, middleware, scripting language, and/or microcode, the program code or code segments to perform the necessary tasks can be stored in a machine-readable medium such as a storage medium. A code segment or machine-executable instruction can represent a procedure, a function, a subprogram, a program, a routine, a subroutine, a module, a software package, a script, a class, or any combination of instructions, data structures, and/or program statements. A code segment can be coupled to another code segment or a hardware circuit by passing and/or receiving information, data, arguments, parameters, and/or memory contents. Information, arguments, parameters, data, etc. can be passed, forwarded, or transmitted via any suitable means including memory sharing, message passing, ticket passing, network transmission, etc.

For a firmware and/or software implementation, the methodologies can be implemented with modules (e.g., procedures, functions, and so on) that perform the functions described herein. Any machine-readable medium tangibly embodying instructions can be used in implementing the methodologies described herein. For example, software codes can be stored in a memory. Memory can be implemented within the processor or external to the processor. As used herein the term “memory” refers to any type of long term, short term, volatile, nonvolatile, or other storage medium and is not to be limited to any particular type of memory or number of memories, or type of media upon which memory is stored.

Moreover, as disclosed herein, the term “storage medium”, “storage” or “memory” can represent one or more memories for storing data, including read only memory (ROM), random access memory (RAM), magnetic RAM, core memory, magnetic disk storage mediums, optical storage mediums, flash memory devices and/or other machine-readable mediums for storing information. The term “machine-readable medium” includes but is not limited to portable or fixed storage devices, optical storage devices, wireless channels, and/or various other storage mediums capable of storing that contain or carry instruction(s) and/or data.

While the principles of the disclosure have been described above in connection with specific apparatuses and methods, it is to be clearly understood that this description is made only by way of example and not as limitation on the scope of the disclosure.

What is claimed:

1. A computer-implemented method comprising:
accessing a set of training data comprising labeled examples that are protein sequences with known activity levels;
generating, using a large language model, synthetic examples by incorporating each possible amino acid mutation into the labeled examples;
selecting, based on a predetermined cutoff, a negative subset of the synthetic examples that comprise at least one amino acid mutation with a lowest probability of being incorporated into a labeled example;
pairing the negative subset of the synthetic examples with respective labeled examples to generate an augmented training dataset comprising pairs of contrastive examples;
training, using the set of training data and the augmented training dataset, a machine learning model to predict activity of a protein sequence, wherein the training is an iterative process that comprises:
(a) feeding a portion or an entirety of the set of training data into the machine learning model, performing computations to generate predictions, comparing the predictions to the known activity levels using a loss function in order to quantify error or loss of the machine learning model,
(b) feeding a portion or an entirety of the augmented training dataset into the machine learning model, performing computations to generate predictions, comparing the predictions for contrast using a contrastive function in order to quantify error or loss of the machine learning model,
(c) adjusting parameters of the machine learning model that jointly minimizes a sum of the loss function and the contrastive function, and
(d) repeating (a), (b), and (c) for a number of iterations or epochs; and
outputting the trained machine learning model.

2. The computer-implemented method of claim 1, wherein the large language model is pretrained using a masked marginal approach training scheme.

3. The computer-implemented method of claim 1, wherein generating the synthetic examples further comprises:
in an iterative process starting at the first amino acid position of the labeled example:
introducing a mask token,
incorporating each possible amino acid mutation into the masked position,
predicting the probabilities for each possible amino acid mutation being incorporated based on the surrounding amino acids of the labeled example, and
repeating the iterative process at the second and subsequent amino acid positions of the labeled example;
outputting the synthetic examples, wherein each of the synthetic examples comprises at least one possible amino acid mutation;
sorting, based on the probabilities for each possible amino acid mutation, the synthetic examples; and
labeling the labeled example and the negative subset of the synthetic examples with labels indicating the labeled example has higher activity compared to the negative subset of the synthetic examples.

4. The computer-implemented method of claim 3, wherein each possible amino acid mutation comprises substitutions, deletions, insertions, or any combination thereof that involves at least one original amino acid in the labeled example.

5. The computer-implemented method of claim 4, wherein each possible amino acid mutation comprises performing a substitution.

6. The computer-implemented method of claim 4, wherein each possible amino acid mutation comprises substituting the at least one original amino acid, inserting one or more amino acids, or any combination thereof with one or more amino acids selected from a list including alanine, cysteine, aspartic acid, glutamic acid, phenylalanine, glycine, histidine, isoleucine, arginine, lysine, leucine, methionine, asparagine, proline, glutamine, serine, threonine, valine, tryptophan, and tyrosine.

7. The computer-implemented method of claim 1, wherein the negative subset of the synthetic examples are highly likely to have reduced activity compared to the labeled example.

8. A system comprising:
one or more data processors; and
a non-transitory computer readable medium storing instructions which, when executed on the one or more data processors, cause the one or more data processors to perform operations comprising:
accessing a set of training data comprising labeled examples that are protein sequences with known activity levels;
generating, using a large language model, synthetic examples by incorporating each possible amino acid mutation into the labeled examples;
selecting, based on a predetermined cutoff, a negative subset of the synthetic examples that comprise at least one amino acid mutation with a lowest probability of being incorporated into a labeled example;
pairing the negative subset of the synthetic examples with respective labeled examples to generate an augmented training dataset comprising pairs of contrastive examples;
training, using the set of training data and the augmented training dataset, a machine learning model to predict activity of a protein sequence, wherein the training is an iterative process that comprises:
(a) feeding a portion or an entirety of the set of training data into the machine learning model, performing computations to generate predictions, comparing the predictions to the known activity levels using a loss function in order to quantify error or loss of the machine learning model,
(b) feeding a portion or an entirety of the augmented training dataset into the machine learning model, performing computations to generate predictions, comparing the predictions for contrast using a contrastive function in order to quantify error or loss of the machine learning model,
(c) adjusting parameters of the machine learning model that jointly minimizes a sum of the loss function and the contrastive function, and
(d) repeating (a), (b), and (c) for a number of iterations or epochs; and
outputting the trained machine learning model.

9. The system of claim 8, wherein the large language model is pretrained using a masked marginal approach training scheme.

10. The system of claim 8, wherein generating the synthetic examples further comprises:
in an iterative process starting at the first amino acid position of the labeled example:
introducing a mask token,
incorporating each possible amino acid mutation into the masked position,
predicting the probabilities for each possible amino acid mutation being incorporated based on the surrounding amino acids of the labeled example, and
repeating the iterative process at the second and subsequent amino acid positions of the labeled example;
outputting the synthetic examples, wherein each of the synthetic examples comprises at least one possible amino acid mutation;
sorting, based on the probabilities for each possible amino acid mutation, the synthetic examples; and
labeling the labeled example and the negative subset of the synthetic examples with labels indicating the labeled example has higher activity compared to the negative subset of the synthetic examples.

11. The system of claim 10, wherein each possible amino acid mutation comprises substitutions, deletions, insertions, or any combination thereof that involves at least one original amino acid in the labeled example.

12. The system of claim 11, wherein each possible amino acid mutation comprises performing a substitution.

13. The system of claim 11, wherein each possible amino acid mutation comprises substituting the at least one original amino acid, inserting one or more amino acids, or any combination thereof with one or more amino acids selected from a list including alanine, cysteine, aspartic acid, glutamic acid, phenylalanine, glycine, histidine, isoleucine, arginine, lysine, leucine, methionine, asparagine, proline, glutamine, serine, threonine, valine, tryptophan, and tyrosine.

14. The system of claim 8, wherein the negative subset of the synthetic examples are highly likely to have reduced activity compared to the labeled example.

15. A computer-program product tangibly embodied in a non-transitory machine-readable medium, including instructions configured to cause one or more data processes to perform operations comprising:
accessing a set of training data comprising labeled examples that are protein sequences with known activity levels;
generating, using a large language model, synthetic examples by incorporating each possible amino acid mutation into the labeled examples;
selecting, based on a predetermined cutoff, a negative subset of the synthetic examples that comprise at least one amino acid mutation with a lowest probability of being incorporated into a labeled example and wherein the negative subset of the synthetic examples are highly likely to have reduced activity compared to the labeled example;
pairing the negative subset of the synthetic examples with respective labeled examples to generate an augmented training dataset comprising pairs of contrastive examples;
training, using the set of training data and the augmented training dataset, a machine learning model to predict activity of a protein sequence, wherein the training is an iterative process that comprises:
(a) feeding a portion or an entirety of the set of training data into the machine learning model, performing computations to generate predictions, comparing the predictions to the known activity levels using a loss function in order to quantify error or loss of the machine learning model,
(b) feeding a portion or an entirety of the augmented training dataset into the machine learning model, performing computations to generate predictions, comparing the predictions for contrast using a contrastive function in order to quantify error or loss of the machine learning model,
(c) adjusting parameters of the machine learning model that jointly minimizes a sum of the loss function and the contrastive function, and
(d) repeating (a), (b), and (c) for a number of iterations or epochs; and
outputting the trained machine learning model.

16. The computer-program product of claim 15, wherein the large language model is pretrained using a masked marginal approach training scheme.

17. The computer-program product of claim 15, wherein generating the synthetic examples further comprises:
in an iterative process starting at the first amino acid position of the labeled example:
introducing a mask token,
incorporating each possible amino acid mutation into the masked position,
predicting the probabilities for each possible amino acid mutation being incorporated based on the surrounding amino acids of the labeled example, and
repeating the iterative process at the second and subsequent amino acid positions of the labeled example;
outputting the synthetic examples, wherein each of the synthetic examples comprises at least one possible amino acid mutation;
sorting, based on the probabilities for each possible amino acid mutation, the synthetic examples; and
labeling the labeled example and the negative subset of the synthetic examples with labels indicating the labeled example has higher activity compared to the negative subset of the synthetic examples.

18. The computer-program product of claim 17, wherein each possible amino acid mutation comprises substitutions, deletions, insertions, or any combination thereof that involves at least one original amino acid in the labeled example.

19. The computer-program product of claim 18, wherein each possible amino acid mutation comprises performing a substitution.

20. The computer-program product of claim 18, wherein each possible amino acid mutation comprises substituting the at least one original amino acid, inserting one or more amino acids, or any combination thereof with one or more amino acids selected from a list including alanine, cysteine, aspartic acid, glutamic acid, phenylalanine, glycine, histidine, isoleucine, arginine, lysine, leucine, methionine, asparagine, proline, glutamine, serine, threonine, valine, tryptophan, and tyrosine.
